(19) United States
(12) Patent Application Publication
(10) Pub. No.: US 2006/0095072 A1
(43) Pub. Date: May 4, 2006

(54) Field circulatory constriction device

(75) Inventors: Carl TenBrink, Huntington Beach, CA (US); David Stocks, Long Beach, CA (US); Tim Frink, Fountain Valley, CA (US)

Correspondence Address: INSKEEP INTELLECTUAL PROPERTY GROUP, INC, 2281 W. 190TH STREET, SUITE 200, TORRANCE, CA, 90504, US

(73) Assignee: Sonos Models, Inc.

(21) Appl. No.: 10/977,265

(22) Filed: Oct. 29, 2004

(51) Int. Cl. A61B 17/00

(52) U.S. Cl. 606/201

(57) ABSTRACT

A field circulatory constriction device suitable for self-application by an injured person in order to stop arterial blood loss in an injured limb includes a strap and a latch for coupling the strap around the limb. A buckle is provided for cinching the coupled strap to the limb and a block and tackle mechanism is provided for increasing the pressure of the cinch strap onto the limb.

### BACKGROUND OF THE INVENTION

The present invention is generally directed to a tourniquet for stopping blood loss from an injured limb and is more particularly directed to a tourniquet for use in emergency situations in which a user is able to apply the tourniquet to his or her own arm or leg and occlude blood flow with the use of a single hand.

Loss of blood in emergency situations and particularly in military confrontations is a major cause of death. In these situations, the injured person often does not have access to immediate medical assistance. It is also well known that the use of a tourniquet to stop blood loss provides temporary first aid treatment until medical assistance is available.

It should be quite apparent that in these situations time is of the essence and thus one handed simple and expedient operation of such an infield circulatory constriction device must be efficiently effected.

Many attempts have been made to develop tourniquets for such in-field use. For example, in U.S. Pat. No. 4,243,039, there is disclosed an emergency tourniquet consisting of a strap and a ratchet-type tensioning device, including a tensional indicating device and a pointer intended to be set by the user to indicate the time of tourniquet application.

Other mechanical restricting devices, such as, for example, disclosed in U.S. Pat. No. 5,628,723, utilize an emergency bandage tightly wrapped around the limbs of pressure dressing and may be used as a tourniquet by using a windless to twist the wrapped bandage and generate sufficient inward radial pressure on the limb to stop arterial blood flow. These prior devices have not proved to be effective or reliable.

On the other hand, pneumatic tournament cuffs have been proven to be effective for stopping arterial blood flow. However, such pneumatic tournament cuffs require a source of pressurized gas to inflate the bladder and infield sources of pressure such as hand pumps or bulbs are typically difficult to handle and may not provide a rapid enough response time in order to effectively stop arterial blood loss.

### OBJECTS AND SUMMARY OF THE INVENTION

A field circulatory constriction device in accordance with the present invention suitable for self application by an injured person in order to stop arterial blood flow or loss in an injured limb generally includes a strap, a latch for coupling the strap around a limb, and a buckle for cinching the coupled strap onto the limb. This mechanical action can be performed in a very brief period of time.

In addition, a block and tackle mechanism is provided for increasing the pressure of the cinched strap onto the limb. Again, this mechanical configuration provides for a rapid and positive action in order to stop arterial blood loss. More particularly, the block and tackle mechanism may include a moving block attached to one end of the strap and a stationary block attached to the latch. A plurality of running lines extend between the moving and stationary blocks and a hauling line extends to the stationary block.

Still more particularly, the device in accordance with the present invention may include a one-way cord locking mechanism disposed at the stationary block for adjusting the hauling line. The one-way cord locking mechanism preferably includes a one-way cord lock that allows a tensioning cord to be pulled through in one direction, but not in another. The tensioning cord is connected to the hauling line, thus drawing the moving block toward the stationary block to constrict the strap about the limb.

More particularly, the one-way cord lock comprises two geared surfaces, biased towards each other to engage the tensioning cord.

Still more particularly, the buckle in accordance with the present invention includes a hook for engaging the latch and the device preferably includes a sleeve for enclosing a block and tackle mechanism to prevent entry of dirt and/or debris.

### BRIEF DESCRIPTION OF THE DRAWINGS

FIG. 1 is a perspective view of a tourniquet or a field circulatory constriction device in accordance with the present invention;

FIG. 2 is a perspective view of the device in accordance with the present invention ready for surrounding a limb;

FIG. 3 is a perspective view similar to that shown in FIG. 2 showing the device latched around the limb and cinched;

FIG. 4 is a cross sectional view of the device in accordance with the present invention showing a strap cinched around a limb;

FIG. 5 is a more detailed view illustrating components of the block and tackle mechanism showing a moving block, a stationary block, and a cord-locking mechanism;

FIG. 6 is a perspective view of a sleeve according to the present invention; and

FIGS. 7A and 7B illustrate a cross-section of a movable block according to the present invention.

### DETAILED DESCRIPTION OF THE INVENTION

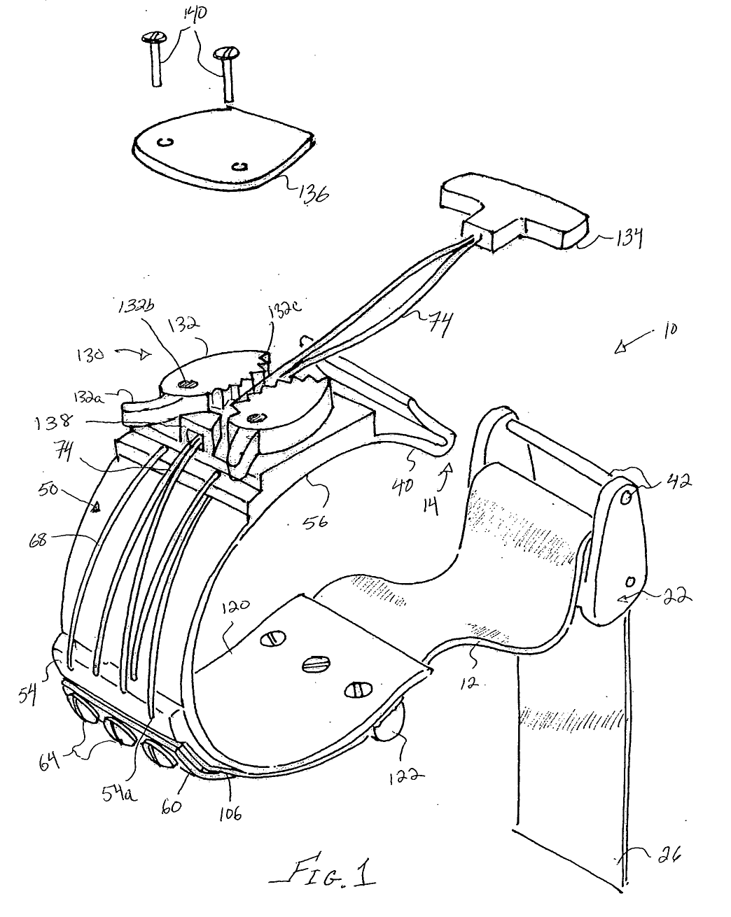

With reference to FIGS. 1-4, there is shown a field circulatory constriction device, or tourniquet, 10 in accordance with the present invention which is suitable for self-application by an injured person in order to stop arterial blood loss in an injured limb.

As shown, the device 10 generally includes a strap 12 made from any suitable flexible material and a hook 40 operable for coupling the strap 12 around a users limb such as, for example, an arm or a leg.

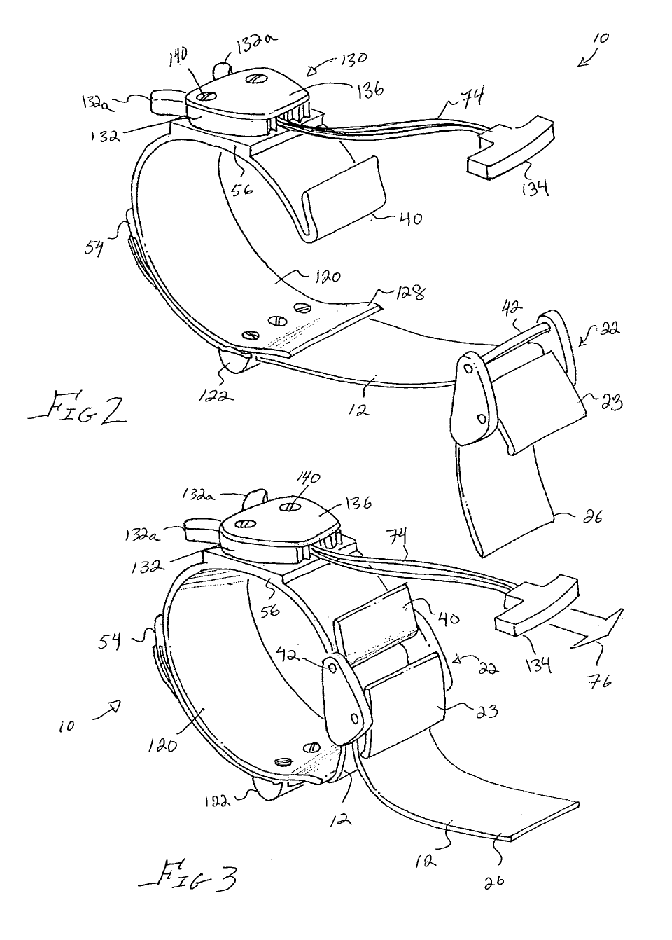

A buckle 22 is provided for cinching a coupled strap 12, see FIG. 3, by pulling on an end 26 of the strap 12. A locking head 30 of clamp 23 engages the strap 12 and forces the strap 12 against a backing plate 32. The angle of the locking head 30 in relation to the backing plate 32 allows the strap 12 to be pulled through the buckle 22 in one direction (e.g. away from the device 10) but not in the opposite direction unless the clamp is released. This arrangement prevents loosening of the strap 12 from around the limb (not shown), as best seen in FIG. 4. Note that the clamp 23 is preferably be biased in an engaged position by, for example a spring as represented by the arrow 177.

In a preferred embodiment, the locking head 30 is comprised of a set of locking teeth or a locking tooth disposed on the clamp. And when the clamp 23 is a biased clamp, the teeth are biased to engage the flexible shaft. In this fashion, the clamp allows movement of the strap 12 through the buckle 22 when the user desires to cinch the device around the limb but does not allow the strap 12 to return through the buckle 22 since movement of the strap will be inhibited by the teeth. In order to allow the strap 12 to return through the buckle, the clamp 23 must be released or urged against the bias to remove the teeth from engaging the strap 12.

It is evident, particularly as shown in FIG. 2 and FIG. 4, that the device 10 is easily positioned around the limb 18 and latched by way of a hook 40 and pin 42 arrangement. The hook 40 includes a protrusion or bump 175 that serves to retain the pin 42 in position in the hook 40.

After coupling the hook 40 and pin 42, as shown in FIG. 4, pulling of the strap end 26 in a direction indicated by the arrow 46 cinches the device 10 around the limb.

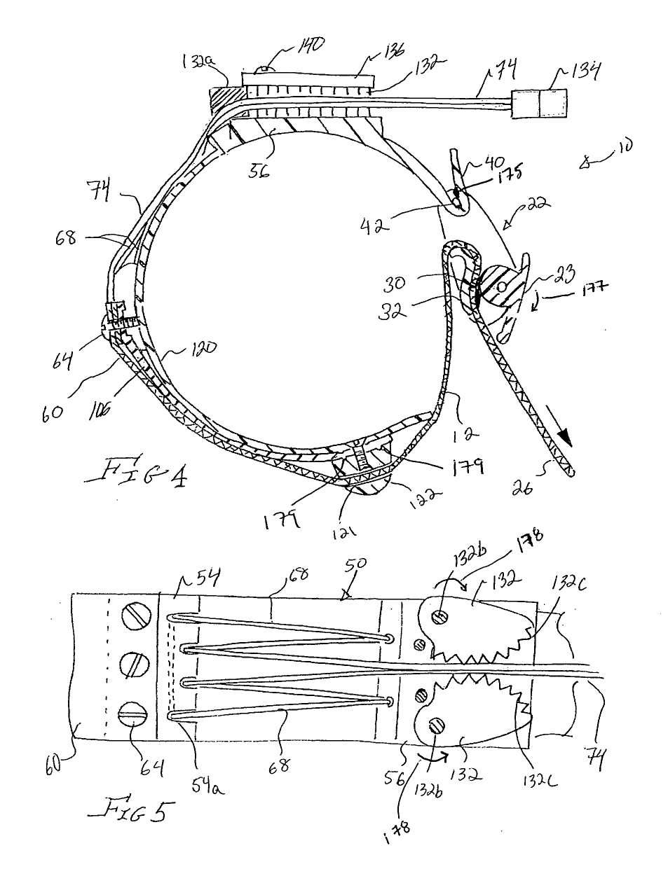

As shown in FIG. 1, and more particularly in FIG. 5, a block and tackle mechanism 50 is provided for increasing pressure of the cinch strap 12 about the limb. More particularly, the block and tackle mechanism 50 includes a moving block 54, a stationary block 56, the stationary block being attached to a strap end 60 in a conventional manner such as, for example, by rivets 64. The stationary block 56 is affixed to the latch 14 by way of the hook 40.

The block and tackle mechanism 50 operates in a conventional manner, including a plurality of running lines 68 and a hauling line 74. Both the running lines 68 and the hauling line 74 are preferably flexible connecting members such as non-elastic plastic chords, rope, or similar structures. The running lines 68 and the hauling line 74 may be separate lines or may optionally be a single, continuous line. In a preferred embodiment, a single line is used for both the running lines 68 and the hauling line 74, as best seen in FIG. 5.

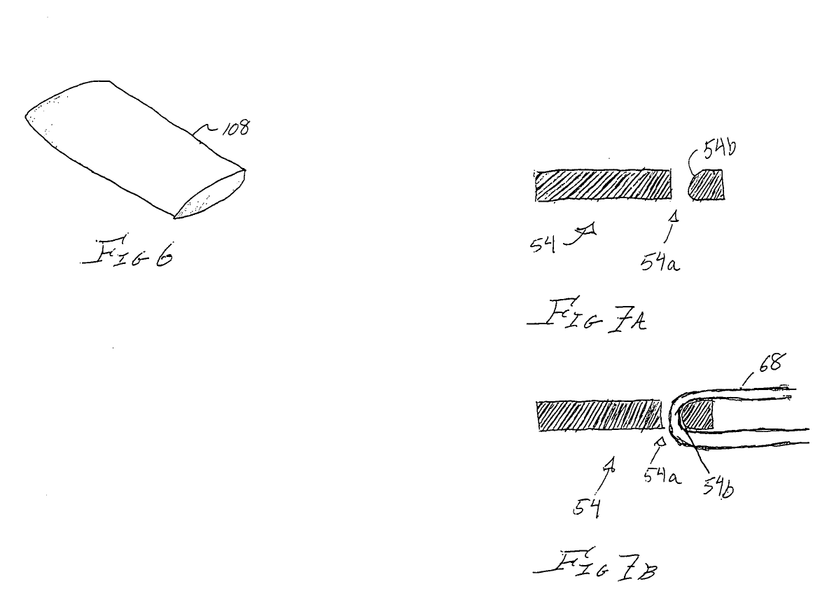

The running lines 68 pass back and forth between the moving block 54 and the stationary block 56. Both blocks 54 and 56 have apertures, such as the apertures 54a as seen in FIGS. 7A and 7B. Apertures 54a include a rounded portion 54b on at least a portion of the aperture 54a so as to prevent sharp edges of the blocks 54 or 56 from rubbing on and wearing down the lines 68 and 74. In this respect, the lifespan of the constriction device 10 may be greatly increased, as wear on the lines 68 and 74 are significantly reduced, leading to a more reliable constriction device 10 than the prior art.

Movement or pulling (such as with one hand) of the hauling line 74 in the direction of the arrow 76 of FIG. 3 causes movement of the moving block 54 toward the stationary block 56, thus causing controlled closure of the device 10 about the limb 18 in order to provide adequate pressure for stopping arterial blood loss from the limb 18.

The hauling line 74 may be locked into place with the one-way line lock 130, disposed on the stationary block 56. The hauling line 74 passes from the moving block 54 through a guide 138 and into contact with the pointed edges 132c of two locking member 132. Each locking member 132 is pivotally mounted to the stationary block 56 with screws 140 positioned through apertures 132b under cover 136. Further, each locking member 132 is biased to pivot (as shown by arrow 178) in the direction of the center of the stationary block 56, towards each other and the hauling line 74.

Due to their oblong shape and the configuration of the pointed edges 132c, the locking members allow the hauling line 74 to be pulled through the one-way lock 130 in the direction of the arrow in FIG. 3, away from the device 10. Tension on the hauling line 74 in the opposite direction (towards the moving block 54) causes the locking members 132 to pivot closer to the hauling line 74, further engaging the hauling line 74 and preventing movement. In this respect, the one-way lock 130, allows the hauling line 74 to move in one direction only when pulled by the handle 134.

If a user desires to release the hauling line 74 to decrease the overall tension caused by the constriction device 10, levers 132a on the ends of the locking members 132 may be squeezed, causing the pointed edges 132c of the locking members to pivot away from the hauling line 74. With the frictional force of the locking members 74 disengaged, the hauling line 74 is free to move in either direction. Typically, the hauling line 74 moves in the direction of the connected moving block 54 in this situation due to a directional bias of the moving block 54 away from the stationary block 56. As best seen in FIG. 4, an elastic member 106 is connected to the moving block 54 and fixture 122, biasing the moving block 54 in the direction of the fixture 122. Since the hauling line 74 is coupled to the moving block 54, disengaging the locking member 132 causes the hauling line 74 to move towards the moving block 54, loosening running lines 68, as best seen in FIG. 5.

As best seen in FIG. 4, fixture 122 includes a slot 121 which allows the strap 12 to freely pass through. This slot 121 allows the strap 12 to maintain alignment with the guard 120, preventing the strap 12 from slipping to the side while increasing tension. In this respect, the slot 121 reduces the risk of improper usage of the constriction device 10, especially in an emergency situation.

In addition, it is noted that the elastic member 106 includes two bumps or ridges 179 that are received in corresponding receptacles of fixture 122. These bumps or ridges 179 may extend substantially the width of said elastic member 106. These bumps or ridges 179 facilitate the retention of the elastic member 106 by the fixture 122.

Preferably, a flexible sleeve 108 having a tube-like shape, seen in FIG. 6, may be positioned over the lines 68 and 74, covering at least a portion of the block and tackle mechanism 50 to prevent dirt and debris from entering and possibly interfering with operation of the constriction device 10. The sleeve 108 may be composed of fabric such as cloth, rubber material such as neoprene, or other flexible materials.

Although the one-way line lock 130 is a preferred embodiment of the present invention, any mechanism may be used to selectively lock and tension the hauling line.

In addition, a guard 120 may be provided and coupled to the strap 12 by way of a fixture 122 and attached to the stationary block 56 in order to provide a spacing between the hook 40 and end 128 of the guard 120, see FIG. 2, and thus facilitate one-handed positioning of the device 10 around the limb 18. The guard 120 may be composed of semi-rigid material such as a flexible plastic. The semi-rigid nature of the guard 120 prevents collapse of the device, which may otherwise interfere with rapid latching of the pin 42 with the hook 40, while also preventing the skin or clothes of the user's limbs from becoming pinched by the constriction device 10.

In operation, a user places the constriction device 10 around a limb and latches pin 42 on hook 40. The protrusion 175 on the hook 40 serves to retain the pin 42 (and thus the buckle 42) in the hook 40. This is an added safety measure. Next, the user pulls down on the end 26 of strap 12, tightening the constriction device 10 around the limb. Finally, the user adjust the final tension of the constriction device 10 by pulling on handle 134 of hauling line 74 to increase tension or releasing the hauling line 74 with levers 132a to relieve tension. Thus, the present invention allows a user to easily achieve a desired tightness around a limb. Further, the ease of use of the present invention allows a user to engage the constriction device with only one hand, making the present invention ideal for self-use in an emergency.

Although there has been hereinabove described a specific field circulatory constriction device in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A constriction device comprising:
a strap;
a buckle coupled with said strap;
a hook device sized and shaped to engage a portion of said buckle;
a block and tackle mechanism coupled to said hook device and said strap; and
wherein said block and tackle mechanism is configured to vary the distance between said hook device and an end of said strap.

2. The constriction device of claim 1 wherein said block and tackle mechanism comprises:
a first block connected to one said end of said strap;
a second block connected to said hook device; and
a flexible connecting member.

3. The constriction device of claim 2 wherein said flexible connecting member is fixed to said first block and slideably coupled to said second block.

4. The constriction device of claim 3 wherein said flexible member is a cord.

5. The constriction device of claim 3 further comprising a lock disposed on said second block, said lock configured to selectively engage said flexible connecting member.

6. The constriction device of claim 5 wherein said lock includes an open state and a closed state.

7. The constriction device of claim 6 wherein said open state allows said flexible connecting member to move through said lock in two directions and said closed state allows said flexible connecting member to move through said lock in one direction.

8. The constriction device of claim 3 wherein said first block and said second block include apertures sized and shaped to allow said flexible connecting member to pass through; at least a portion of said apertures having a rounded wall.

9. The constriction device of claim 1 wherein said block and tackle mechanism is substantially covered by a sleeve.

10. The constriction device of claim 2 further comprising a guard fixed to said second block.

11. The constriction device of claim 10 wherein said guard is sized and shaped to prevent pinching of skin or clothing.

12. A method of constricting a human limb comprising:
providing a constriction device having a strap; a coupler configured to couple a first area of said strap with a second area of said strap; and a block and tackle mechanism coupled to said strap;
coupling a first portion of said strap with a second portion of said strap to encircle a limb; and
engaging said block and tackle mechanism to increase tension around said limb.

13. The method of claim 12 wherein only one hand is needed to couple and engage said constriction device.

14. The method of claim 12 wherein said coupler is a hook fixed to said first portion of said strap and a buckle disposed on a second portion of said strap.

15. The method of claim 12 wherein engaging said block and tackle mechanism further comprises pulling a hauling line.

16. The method of claim 12 wherein said block and tackle mechanism includes a one-way lock, configured to engage a hauling line.

17. A constriction device comprising:
a flexible member;
a buckle device engaged with said flexible member;
a hook device sized and shaped to engage a portion of said buckle device;
a block and tackle mechanism coupled to said hook device and said flexible member; and
wherein said block and tackle mechanism is configured to vary the distance between said hook device and said flexible member.

18. The constriction device of claim 17 wherein said flexible member is a strap.

19. The constriction device of claim 17 further comprising:
a first block connected to one end of said flexible member having a first set of apertures;
a second block connected to said hook device having a second set of apertures; and
a connecting line; and
wherein said first set of apertures and said second set of apertures include rounded walls; said connecting line passing through said first set of apertures and said second set of apertures.

20. The constriction device of claim 17, wherein said hook device includes a protrusion for securing the engagement of said portion of said buckle device in said hook device.

21. The constriction device of claim 1, wherein said hook device includes a protrusion for securing engagement of said portion of said buckle.

22. The constriction device of claim 17, wherein said buckle device includes a clamp for slidable engagement with said flexible member so as to allow selective constriction of said device.

23. The constriction device of claim 22, wherein said clamp includes a plurality of teeth positioned on said claim so as to allow movement of said flexible member in one direction and substantially inhibit movement of said flexible member in an opposite direction.

24. The constriction device of claim 23, wherein said clamp is biased so to cause engagement of said teeth with said flexible member.
